(19) United States
(12) Patent Application Publication
(10) Pub. No.: US 2025/0178989 A1
(43) Pub. Date: Jun. 5, 2025

(54) COMPOSITIONS AND METHODS FOR MAKING HFO-153-10MZZ AND HFO-153-10MCZZ

(71) Applicant: THE CHEMOURS COMPANY FC, LLC, WILMINGTON, DE (US)

(72) Inventors: VIACHESLAV A. PETROV, HOCKESSIN, DE (US); DREW RICHARD BRANDT, WEST CHESTER, PA (US); JASON R. JUHASZ, HOCKESSIN, DE (US); LUKE DAVID SIMONI, WILMINGTON, DE (US); JONATHAN P. STEHMAN, WILMINGTON, DE (US); STEPHAN M. BRANDSTADTER, PHILADELPHIA, PA (US)

(73) Assignee: THE CHEMOURS COMPANY FC, LLC, WILMINGTON, DE (US)

(21) Appl. No.: 18/840,975

(22) PCT Filed: Feb. 24, 2023
(86) PCT No.: PCT/US2023/013807

§ 371 Date: Aug. 23, 2024

Related U.S. Application Data
(60) Provisional application No. 63/313,769, filed on Feb. 25, 2022.
(60) Provisional application No. 63/444,716, filed on Feb. 10, 2023.

(51) Int. Cl. C07C 17/25; C07C 21/18

(52) U.S. Cl. CPC C07C 17/25; C07C 21/18

(57) ABSTRACT

A method of producing a fluoroolefin includes hydrodechlorinating a compound of formula (1), CₙF₂ₙ₊₁CX₁═CClC₄₋ₙF₉₋₂ₙ, where n is 1 or 2 and where X₁ is H or Cl, in the presence of a first catalyst in an amount sufficient to form a composition including a compound of formula (2), CₙF₂ₙ₊₁CH═CHC₄₋ₙF₉₋₂ₙ. In some embodiments, the method also includes contacting CF₃CCl═CClCF₃ or CF₃CCl═CHCF₃ with CF₂═CF₂ in the presence of a second catalyst in an amount sufficient to form a composition including the compound of formula (1). In other embodiments, the method also includes coupling C₂F₅CCl₃ under conditions sufficient to form a composition including the compound of formula (1). In other embodiments, the method further includes chlorinating C₂F₅CH₃ in the presence of a second catalyst in an amount sufficient to form a composition comprising C₂F₅CCl₃.

### CROSS REFERENCE TO RELATED APPLICATION

This is a national stage filing under 35 U.S.C. 371 of International Application No. PCT/US2023/013807 and claims the benefit of priority of U.S. Provisional Application No. 63/313,773, filed Feb. 25, 2022, and U.S. Provisional Application No. 63/444,716, filed Feb. 10, 2023. The disclosures of the foregoing commonly assigned applications are hereby incorporated by reference.

### FIELD

The present disclosure is directed to the production of fluorinated alkene compounds. More specifically, the present disclosure is directed to the production of the hydrofluoroolefins (HFOs) E-1,1,1,2,2,5,5,6,6,6-decafluoro-3-hexene (HFO-153-10mczz; C₂F₅CH═CHCF₂CF₃) and 1,1,1,4,4,5,5,6,6,6-decafluoro-2-hexene (HFO-153-10mzz; C₃F₇CH═CHCF₃).

### BACKGROUND

A growing public awareness of the environmental impacts from the extraction, transportation, and use of fossil fuels is motivating a new environmental sustainability driver in the form of regulations and reduction in output of CO₂ equivalents in the atmosphere. New working fluids with a low global warming potential (GWP) and a low ozone depletion potential (ODP) for both existing and new applications in thermal management segments will need to adhere to these new regulations.

### SUMMARY

In an embodiment, a method of producing a fluoroolefin includes hydrodechlorinating a compound of formula (1), CₙF₂ₙ₊₁CX₁═CClC₄₋ₙF₉₋₂ₙ(1) wherein n is 1 or 2; and wherein X₁ is H or Cl; in the presence of a first catalyst in an amount sufficient to form a composition comprising a compound of formula (2), CₙF₂ₙ₊₁CH═CHC₄₋ₙF₉₋₂ₙ(2).

The present disclosure includes the following aspects and embodiments:

In one embodiment, disclosed herein are methods of producing a fluoroolefin. The methods described herein comprise: hydrodechlorinating a compound of formula (1), CnF2n+1CX1=CClC4−nF9−2n (1) wherein n is 1 or 2; and wherein X₁ is H or Cl; in the presence of a first catalyst in an amount sufficient to form a composition comprising a compound of formula (2), CnF2n+1CH═CHC4−nF9−2n (2).

According to the forgoing embodiment, also disclosed herein are methods wherein the hydrodechlorinating occurs in a vapor phase.

According to any of the foregoing embodiments, also disclosed herein are methods wherein the first catalyst comprises a palladium catalyst.

According to any of the foregoing embodiments, also disclosed herein are methods wherein n is 1 and X₁ is Cl.

According to any of the foregoing embodiments, also disclosed herein are methods wherein the compound of formula (2) is unbranched.

According to any of the foregoing embodiments, also disclosed herein are methods further comprising contacting CF₃CCl═CClCF₃ with CF₂═CF₂ in the presence of a second catalyst in an amount sufficient to form a composition comprising the compound of formula (1).

According to the forgoing embodiment, also disclosed herein are methods wherein the contacting occurs in a vapor phase.

According to any of the foregoing embodiments, also disclosed herein are methods wherein the second catalyst comprises aluminum chlorofluoride.

According to any of the foregoing embodiments, also disclosed herein are methods wherein the aluminum chlorofluoride has the formula AlClₓF₃₋ₓ, wherein x is in the range of 0.05 to 0.3.

According to any of the foregoing embodiments, also disclosed herein are methods wherein the CF₃CCl═CClCF₃ is formed as an intermediate product in a production of Z—CF₃—CH═CH—CF₃, for example, as disclosed in WO 2015/120250, by coupling 1,1,1-trichloro-2,2,2-trifluoroethane (CFC-113a) in the presence of a ruthenium catalyst supported on SiC.

According to any of the foregoing embodiments, also disclosed herein are methods wherein n is 1 and X₁ is H.

According to any of the foregoing embodiments, also disclosed herein are methods further comprising contacting CF₃CCl═CHCF₃ with CF₂═CF₂ in the presence of a second catalyst in an amount sufficient to form a composition comprising the compound of formula (1).

According to the forgoing embodiments, also disclosed herein are methods wherein the contacting occurs in a vapor phase.

According to any of the foregoing embodiments, also disclosed herein are methods wherein the second catalyst comprises SbF₅.

According to any of the foregoing embodiments, also disclosed herein are methods wherein the CF₃CCl═CHCF₃ is formed as an intermediate product in a production of Z—CF₃—CH═CH—CF₃ for example, as disclosed in WO 2015/120250, by contacting CF₃CCl═CClCF₃ with hydrogen in the presence of a catalyst comprising copper.

According to any of the foregoing embodiments, also disclosed herein are methods wherein n is 2.

According to any of the foregoing embodiments, also disclosed herein are methods wherein X₁ is Cl.

According to any of the foregoing embodiments, also disclosed herein are methods further comprising coupling C₂F₅CCl₃ under conditions sufficient to form a composition comprising the compound of formula (1).

According to the forgoing embodiment, also disclosed herein are methods wherein the coupling occurs in a vapor phase.

According to any of the foregoing embodiments, also disclosed herein are methods wherein the coupling is performed at a temperature of about 130° C.

According to any of the foregoing embodiments, also disclosed herein are methods further comprising chlorinating C₂F₅CH₃ in the presence of a second catalyst in an amount sufficient to form a composition comprising C₂F₅CCl₃.

According to any of the foregoing embodiments, also disclosed herein are methods wherein the second catalyst comprises SbF₅.

According to any of the foregoing embodiments, also disclosed herein are methods wherein the C₂F₅CH₃ is formed as a byproduct in a production of CF₃—CF═CH₂.

According to any of the foregoing embodiments, also disclosed herein are compositions formed by any of the foregoing methods.

In some embodiments, the compound of formula (2) comprises E-C₃FCH═CHC₂F₅ (E-1,1,1,2,2,5,5,6,6,6-decafluoro-3-hexene; E-HFO-153-10mczz).

According to certain embodiments a composition for use as a working fluid or heat exchange fluid comprises one of CF₃CCl═CHC₃F₇ (2-chloro-1,1,1,4,4,5,5,6,6,6-decafluoro-2-hexene), CF₃CCl═CClC₃F₇ (2,3-dichloro-1,1,1,4,4,5,5,6,6,6-decafluoro-2-hexene), C₂F₅CCl═CClC₂F₅ (3,4-dichloro-1,1,1,2,2,5,5,6,6,6-decafluoro-3-hexene), CF₃CH═CHC₃F₇ (1,1,1,4,4,5,5,6,6,6-decafluoro-2-hexene; HFO-153-10mzz), alone or in combination with one another, optionally including other HFCs, HFOs, HCFOs.

According to other embodiments a composition for use as a working fluid or heat exchange fluid comprises one or more of the compounds identified in Table 1, alone or in combination with one another, optionally including other HFCs, HFOs, and HCFOs.

The embodiments disclosed herein may be used alone or in various combinations with other embodiments. Other features and advantages of the present invention will be apparent from the following more detailed description, which illustrates, by way of example, the principles of the invention.

### DETAILED DESCRIPTION

Provided are synthesis methods for the production of fluorinated alkenes, which overcome the limitations described above.

Embodiments of the present disclosure, for example, provide methods for the production of fluorinated alkenes. More specifically, the present disclosure provides methods for the production of fluorinated alkenes having a perfluorinated alkyl chain. The resulting fluorinated alkenes are environmentally friendly, exhibiting a low GWP and low ozone depletion potential (ODP), non-flammable, non-conductive, and exhibit low liquid viscosities.

In some embodiments, a compound of formula (1), CₙF₂ₙ₊₁CX₁═CClC₄₋ₙF₉₋₂ₙ(1), wherein n is 1 or 2; and wherein X₁ is H or Cl; is charged to a reactor, heated, and contacted with hydrogen gas, in the presence of a first catalyst. The temperature and pressure of the reactor are maintained at levels sufficient to effect, in the presence of the first catalyst, the formation of a composition comprising a compound of formula (2), CₙF₂ₙ₊₁CH═CHC₄₋ₙF₉₋₂ₙ (2).

In some embodiments, a compound of formula (3), CₙF₂ₙ₊₁CCl₂CCl₂ C₄₋ₙF₉₋₂ₙ (3), wherein n is 1, is hydrodechlorinated in the presence of a catalyst in an amount sufficient to form a composition comprising a compound of formula (2) CₙF₂ₙ₊₁CH═CHC₄₋ₙF₉₋₂ₙ(2).

Methods and compositions of the present disclosure may include one or more of a compound of formula (1) and a compound of formula (2). Each of these compounds has an E isomeric form and a Z isomeric form. As used herein, when the form is unspecified, the composition may include the E isomer, the Z isomer, or any combination thereof.

In some embodiments, the E isomer is preferred. In some embodiments, starting materials and/or method conditions are selected to increase formation of the E isomer over the Z isomer. In some embodiments, the method includes separating the E isomer from the Z isomer.

In some embodiments, the reaction occurs in the vapor phase.

In some embodiments, the first catalyst includes palladium.

In some embodiments, the compounds of formulas (1) and (2) are unbranched.

In some embodiments, n is 1 and X₁ is Cl, and CF₃CCl═CClCF₃ is charged to a reactor, heated, and contacted with CF₂═CF₂, in the presence of a second catalyst. The temperature and pressure of the reactor are maintained at levels sufficient to effect, in the presence of the second catalyst, the formation of a composition including a compound of formula (1).

In some embodiments, the reaction occurs in the vapor phase.

In some embodiments, the second catalyst includes aluminum chlorofluoride.

In some embodiments, the aluminum chlorofluoride has the formula AlClₓF₃₋ₓ, wherein x is in the range of 0.05 to 0.3.

In some embodiments, the CF₃CCl═CClCF₃ is formed as an intermediate product in the production of Z—CF₃CH═CHCF₃ (Z-HFO-1336mzz) by coupling 1,1,1-trichloro-2,2,2-trifluoroethane (CFC-113a) in the presence of a ruthenium catalyst. In some embodiments, the ruthenium catalyst is supported on SiC.

In some embodiments, n is 1 and X₁ is H, and CF₃CCl═CHCF₃ is charged to a reactor, heated, and contacted with CF₂═CF₂, in the presence of a second catalyst. The temperature and pressure of the reactor are maintained at levels sufficient to effect, in the presence of the second catalyst, the formation of a composition including a compound of formula (1).

In some embodiments, the reaction occurs in the vapor phase.

In some embodiments, the second catalyst includes SbF₅.

In some embodiments, the CF₃CCl═CHCF₃ is formed as an intermediate product in the production of Z—CF₃CH═CHCF₃, by contacting CF₃CCl═CClCF₃ with hydrogen in the presence of a catalyst comprising copper. The catalyst comprising copper may be selected from the group consisting of copper on carbon, nickel on carbon, copper and nickel on carbon and copper and palladium on carbon.

In some embodiments, n is 2 and X₁ is Cl, and C₂F₅CCl₃ is charged to a reactor and heated. The temperature and pressure of the reactor are maintained at levels sufficient to effect coupling of the C₂F₅CCl₃, the formation of a composition including a compound of formula (1). In some embodiments, C₂F₅CH₃ is charged to a reactor, heated, and contacted with chlorine gas, in the presence of a second catalyst. The temperature and pressure of the reactor are maintained at levels sufficient to effect, in the presence of the second catalyst, the formation of a composition including C₂F₅CCl₃.

In some embodiments, the reaction occurs in the vapor phase.

In some embodiments, the second catalyst includes SbF₅.

In some embodiments, the C₂F₅CH₃ is formed as a byproduct in a production of CF₃—CF═CH₂ (HFO-1234yf).

In some embodiments, the compound of formula (1) comprises CF₃CCl═CHC₃F₇ (2-chloro-1,1,1,4,4,5,5,6,6,6-decafluoro-2-hexene).

In some embodiments, the compound of formula (1) comprises CF₃CCl═CClC₃F₇ (2,3-dichloro-1,1,1,4,4,5,5,6,6,6-decafluoro-2-hexene).

In some embodiments, the compound of formula (1) comprises C₂F₅CCl═CClC₂F₅ (3,4-dichloro-1,1,1,2,2,5,5,6,6,6-decafluoro-3-hexene; CFO-151-10mcxx).

In some embodiments, the compound of formula (2) comprises CF₃CH═CHC₃F₇ (1,1,1,4,4,5,5,6,6,6-decafluoro-2-hexene; HFO-153-10mzz).

In some embodiments, the compound of formula (2) comprises C₂F₅CH═CHC₂F₅ (1,1,1,2,2,5,5,6,6,6-decafluoro-3-hexene; HFO-153-10mczz).

In some embodiments, C₂F₅CH═CHC₂F₅ is E-C₂F₅CH═CHC₂F₅(E-1,1,1,2,2,5,5,6,6,6-decafluoro-3-hexene; E-HFO-153-10mczz.

In some embodiments, the compound formed by hydrodechlorinating a compound of formula (1) comprises C₂F₅CCl═CHCF₂CF₃H (HCFO-152-10mcxz).

In some embodiments, the compound formed by hydrodechlorinating a compound of formula (1) comprises C₂F₅—CCl═CClC₂F₅(CFO-151-10mcxx).

In some embodiments, the compound formed by hydrodechlorinating a compound of formula (1) comprises C₂F₅—CH═CH—C₂F₅(HFO-153-10mczz).

In some embodiments, the compound formed by hydrodechlorinating a compound of formula (1) comprises a mixture of HCFO-152-10mcxz, CFO-151-10mcxx, and HFO-153-10mczz.

In some embodiments, a reaction step is conducted in a closed system. In some embodiments, the catalyst for a reaction step is a Lewis acid. In some embodiments, the Lewis acid is a strong Lewis acid. In one embodiment, the catalyst is, aluminum chloride (AlCl₃), or antimony pentafluoride (SbF₅), or aluminum chlorofluoride AlClₓF₃₋ₓ or a compound of formula (4), SbClₓF₅₋ₓ. In some embodiments, x may be an integer from 1 to 3. In some embodiments, x may be 0.01 to 0.5. In some embodiments, x may be 0.05 to 0.3. Additional suitable strong Lewis acids may be found in Krespan et al., “The Chemistry of Highly Fluorinated Carbocations”, Chemical Reviews, Vol. 96, pp. 3269-3301, 1996, which is incorporated by reference herein.

The process may be conducted in any reactor suitable for a vapor phase fluorination reaction. The reactor is made of a material that is resistant to the reactants employed. The reactor may be constructed from materials which are resistant to the corrosive effects of hydrogen fluoride such as stainless steel, a Hastelloy® alloy, an Inconel® alloy, a Monel® alloy, gold, gold-lined, or quartz.

The reactions may be conducted batchwise, continuous, semi-continuous or combinations thereof. Suitable reactors include batch reactor vessels and tubular reactors.

In some embodiments, a reaction mixture is heated to a sub-ambient, ambient, or super-ambient temperature. In some embodiments, the reaction mixture is heated to a temperature of −50° C. to 50° C. In some embodiments, the reaction mixture is heated to a temperature of −50° C. to 25° C. In some embodiments, the reaction mixture is heated to a temperature of 50° C. to 100° C. In some embodiments, the reaction mixture is heated to a temperature of 100° C. to 150° C.

In some embodiments, a reaction step is performed at a reactor pressure of 0.1 pound per square inch gauged (psig) (690 Pa) to 300 pounds per square inch gauged (psig) (2.07 MPa). In some embodiments, the reaction step is performed under autogenic pressure.

In some embodiments, a reaction step is conducted in the presence of a solvent. In some embodiments, the solvent is a perfluorinated saturated compound. In some embodiments, the perfluorinated saturated compound may include perfluoropentane, perfluorohexane, cyclic dimer of hexafluoropropene, (mixture of perfluoro-1,2- and perfluoro-1,3-dimethylcyclobutanes), and combinations thereof.

In some embodiments hydrodechlorination is conducted in the presence of a catalyst.

In some embodiments the hydrodechlorination is conducted in the presence of a Pd containing catalyst.

In some embodiments the hydrodechlorination is conducted in the presence of Pd—Cu supported on activated carbon catalyst.

The compound of formula (2) may be isolated and optionally purified prior to use. Suitable uses of a compound of formula (2) may include, but are not limited to, a working fluid in a system utilizing a thermodynamic cycle, a cooling medium, a specialty fluid for thermal management, an immersion cooling fluid, a reactive intermediate, a refrigerant, a heat transfer fluid with or without phase change, a carrier fluid, or a solvent.

The good dielectric properties and suitable boiling point of a compound of formula (2) make it a potential candidate for use as a cooling medium for a lithium-ion battery (LiB) in an automobile.

In other exemplary embodiments, the properties of a compound of formula (2) yield benefits in carrier fluid applications. A compound of formula (2) exhibits good characteristics to enable it to provide traditional carrier fluid behavior for the deposition or removal of soluble compounds, where it readily dissolves, transports, and/or deposits specified media.

In other exemplary embodiments, a compound of formula (2) is used as a solvent for any of a number of various applications. For example, the properties of a compound of formula (2) may yield benefits in solvent cleaning applications. Additional solvent-based of applications for a compound of formula (2) include as a fluid for removal of particulates, greases, oils, and contamination. a compound of formula (2) may also be used as solvents in various applications such as for cleaning (vapor degreasing, flux removal).

In exemplary embodiments, a compound of formula (2) serves as a specialty fluid for thermal management, with slightly elevated boiling temperature ranges, where the product is environmentally friendly (low GWP and ODP), non-flammable, non-conductive, and has low liquid viscosities.

A compound of formula (2) may also be used as a working fluid for immersion cooling, which may be two-phase immersion cooling or single phase immersion cooling.

Two-phase immersion cooling is an emerging cooling technology for the high-performance cooling market as applied to high performance server systems. It relies on the heat absorbed in the process of vaporizing a liquid immersion cooler fluid to a gas. The fluids used in this application must meet certain requirements to be viable in use. For example, the boiling temperature of the fluid should be in the range between 30-75° C. Generally, this range accommodates maintaining the server components at a sufficiently cool temperature while allowing generated heat to be dissipated sufficiently to an external heat sink. Alternatively, the operating temperature of the server, and the immersion cooling system could be raised or lowered, by using an enclosed system and raising or lowering the pressure within the system to raise or lower the boiling point of a given fluid.

Single phase immersion cooling has a long history in computer server cooling. There is no phase change in single phase immersion cooling. Instead, the liquid warms as it circulates through the computer server and or heat exchanger, and then is circulated with a pump to a heat exchanger for cooling prior to returning to the server, thus transferring heat away from the computer server. Fluids used for single phase immersion cooling have the same requirements as those for two-phase immersion cooling, except that the boiling temperatures are typically higher than 30-75° C., to reduce loss by evaporation.

In exemplary embodiments, a compound of formula (2) serves as an immersion cooling fluid having an operating temperature range near ambient temperatures. Embodiments of the present disclosure, for example, in comparison to concepts failing to include one or more of the features disclosed herein, provide an immersion cooling fluid for thermal management which is environmentally friendly (i.e., have a low global warming potential (GWP) and low ozone depletion potential (ODP)).

In exemplary embodiments, the immersion cooling fluid cools a heat generating component by at least partially immersing the heat generating component of a device into the immersion cooling fluid in a liquid state such that heat is transferred from the heat generating component using the immersion cooling fluid. Such devices may include, but are not limited to, high-capacity energy storage devices, electrical components, mechanical components and optical components. Appropriate devices may include, but are not limited to, microprocessors, wafers used to manufacture semiconductor devices, power control semiconductors, electrical distribution switch gear, power transformers, circuit boards, multi-chip modules, packaged and unpackaged semiconductor devices, laser, fuel cells, electrochemical cells and energy storage devices such as batteries.

The opportunities for a compound of formula (2) as a new working fluid exist potentially in a variety of heat transfer applications, including, but not limited to, cooling power electronics, such as, for example, televisions, cell phones, monitors, drones, and avionics devices; battery thermal management in both automotive and stationary systems; powertrains for electronic vehicles; insulated-gate bipolar transistors (IGBTs); electronic devices-data center servers; computer server systems; telecommunication infrastructure; 5G network; displays; military electronics; high temperature mechanical compression heat pumps (HTHPs); Organic Rankine Cycles (ORCs); and anywhere a working fluid provides a medium to transport heat or in applications where passive evaporative cooling exists, such as, for example, heat pipes.

A compound of formula (2) may be used in numerous applications for the transfer of heat, such as, heat transfer fluids or refrigerants. In one embodiment, a compound of formula (2) may be used to transfer heat from an article. The article may be contacted with a heat transfer media including a compound of formula (2).

A compound of formula (2) may be used in various applications including as working fluids. Working fluids provide the medium to transport heat or produce power by mechanical means by expansion. Working fluids are typically in the liquid state at a first region. The working fluid absorbs heat in the first region, vaporizes, and migrates to a second region, having a lower temperature, where it condenses. The working fluid is typically returned to the first region after condensation allowing the heat transfer cycle to be repeated.

Working fluids may be used in conjunction with compression, expansion systems, pumps, or in passive evaporative cooling such as heat pipes or thermosyphons.

During use, the working fluid in a first region is exposed to an elevated (first) temperature causing the working fluid to vaporize, thus absorbing thermal energy. The vaporized working fluid migrates to a second region, which is at a lower (second) temperature than the first region. The working fluid condenses in the second region, releasing the thermal energy, which is transported external to the system. The working fluid is subsequently returned to the first region. The working fluid typically cyclically moves between the first region and the second region, transporting thermal energy between the first region and the second region.

Working fluids are selected to undergo a phase transition from the liquid to the gaseous state over the desired operational temperature range of a system, such as a heat pipe or thermosyphon. In some embodiments, the composition of the working fluids includes a compound of formula (2). In some embodiments, the operational temperature is at least 0° C., at least 10° C., at least 20° C., at least 30° C., at least 40° C., at least 50° C., at least 60° C., at least 70° C., at least 80° C., at least 90° C., at least 100° C., less than 125° C., less than 120° C., less than 110° C., less than 100° C., less than 90° C., less than 75° C., less than 70° C., less than 65° C., less than 60° C., less than 55° C., and combinations thereof.

The ability of the working fluid to transport heat is related to the heat of vaporization of the working fluids. The greater the heat of vaporization of the working fluids the greater amount of energy that the working fluid will absorb during vaporization and transport across the heat pipe to be released during condensation. In some embodiments, a compound of formula (2) may exhibit a heat of vaporization of at least 35 kilojoules per mole (kJ/mol).

Working fluids may also be selected based at least partially on additional material properties. As the working fluids condense and return to the first region workings fluids having a lower viscosity more easily flow between the regions. In some embodiments, a compound of formula (2) may exhibit a viscosity less than water of the same temperature, over the operational temperature range. In some embodiments, a compound of formula (2) may exhibit a viscosity of less than 0.5 centipoise at 55° C.

A compound of formula (2) as a working fluid for heat transfer applications may be selected based at least partially on the surface tension exhibited by the material. For example, in heat pipe applications, working fluids exhibiting high surface tensions may be more easily transported between the hot region and the cool region. In some embodiments, the selection of the wick materials may enhance the rate at which the condensed working fluid is returned to the hot region of the heat pipe. In some embodiments, the working fluids may exhibit a surface tension less than water of the same temperature, over the operational temperature range. In some embodiments, a compound of formula (2) may exhibit a surface tension of less than 64.5 dyne/cm at 70° C., less than 66.3 dyne/cm at 60° C., and/or less than 67.9 dyne/cm at 50° C.

The working fluids may also be selected based at least partially on other thermodynamic properties of the materials. Working fluids exhibiting a lower specific heat and/or a lower thermal conductivity than water at the same temperature may enhance energy transport between the hot region and the cool region of a heat pipe. In some embodiments, the working fluids may exhibit a specific heat of less than 4.2 Joules per gram Kelvin degree. In some embodiments, a compound of formula (2) may exhibit a thermal conductivity of less than 0.6 watts per meter Kelvin degree at 20° C.

The working fluids may also be selected to exhibit a dielectric constant suitable for electrical applications. In general, materials exhibiting a low dielectric constant provide increased electrical isolation of the electrical components immersed therein. In some embodiments, the dielectric constant of the working fluids is less than about 8 over the operational frequency range (0 to 20 GHz). Suitable dielectric working fluids include a compound of formula (2) having a dielectric constant over the operational frequency range (0 to 20 GHz) of less than 7.3, or less than 5.5, or less than 5.0, or less than 4.0, or less than 3.5, or less than 2.7, or less than 2.5, or less than 2.0, or less than 1.9, or less than 1.8, or less than 1.5. Other embodiments include compounds and mixtures having a dielectric constant greater than 1.0 and less than 8.0 or greater than 2.0 and less than 7.3 or greater than 2.5 and less than 5.5 or greater than 3.5 and less than 5.0.

Table 1 shows certain properties relevant for working fluids for a compound of formula (2) compared to other similar compounds.

TABLE 1

| | method = | | | |
|---|---|---|---|---|
| | basis | MP2 | | |
| | set= | 6-311G | liquid at | |
| | dipole | (d, p) | 25° C. | dielec- |
| | moment | polarizability | density | tric |
| Fluid | (Debye) | (a₀³) | (g/cm³) | constant |
| E-C₂F₅CF═CFC₂F₅ | 0.00 | 63.27 | 1.64 | 1.75* |
| (FO-151-12mcyyE) | | | | |
| E-C₂F₅CH═CHC₂F₅ | 0.10 | 62.74 | 1.52 | 1.82* |
| (HFO-153-10mczzE) | | | | |
| C₃F₇CH═CHCF₃ | 0.24 | 62.81 | 1.51 | 2.09* |
| (HFO-153-10mzzE)Z- | | | | |
| (CF₃)₂CFCH═CHCF₃ | 0.20 | 62.66 | 1.49 | 1.85* |
| (HFO-153-10mzzyE) | | | | |
| C₂F₅CF═CFC₂F₅ | 0.43 | 63.45 | 1.64 | 1.59** |
| (FO-151-12mcyZ) | | | | |
| CF₃CH═CHCF₂CF₂Cl | 0.59 | 60.85 | 1.49 | 1.74** |
| E-isomer | | | | |
| CF₃CH═CHCF₂CF₂CF₂Cl | 0.84 | 71.90 | 1.53 | 1.94** |
| E-isomer | | | | |
| CF₃CH═CHCF₂CFClCF₃ | 0.59 | 72.14 | 1.74 | 2.09** |
| E-isomer | | | | |
| CF₃CH═CHCF₂CFClCF₃ | 0.68 | 61.66 | 1.57 | 2.28** |
| E-isomer | | | | |
| C₄F₉CH═CFH | 1.48 | 63.26 | 1.55 | 3.77** |
| (HFO-153-10ze)Z-isomer | | | | |
| E-CF₃CCl═CFCl | 0.98 | 50.72 | 1.53 | 2.67** |
| (CFO-1214xbE) | | | | |
| CF₃CF═CCl₂ (CFO- | 1.26 | 50.90 | 1.53 | 3.42** |
| 1214ya) | | | | |
| C₂F₅CH═CFC₂F₅ | 1.41 | 62.88 | 1.61 | 3.46** |
| (HFO-152-11mcyz) E- | | | | |
| isomer | | | | |
| BrCF₂CF₂CH═CH₂ | 2.16 | 57.52 | 1.73 | 4.81** |
| CF₃CCl═CFCl, Z-isomer | 1.78 | 50.30 | 1.53 | 5.45** |
| (CFO-1214xbZ) | | | | |
| Z-HCF₂CF═CHCl | 1.74 | 41.37 | 1.32 | 7.28** |
| (HCFO-1233ydZ) Z-isomer | | | | |

*measured using ASTM D924
**calculated using conventional methods known in the art

Additional additives may be added to the working fluid. Suitable additives include linear hydrocarbons, linear halocarbons, cyclic hydrocarbons, cyclic halocarbons, heptafluorocyclopentane, alcohols (e.g., methanol, ethanol, isopropanol), ethers, halogenated ethers, ketones, and halogenated ketones.

Examples of suitable additives include pentane (bp 36° C.), hexane (bp 69° C.), heptane (bp 98° C.), octane (bp 125° C.), cyclopentane (bp 49° C.), cyclohexane (bp 80° C.), cycloheptane (bp 118° C.), methyl cyclobutane (bp 39° C.), and methylcyclopentane (bp 72° C.). Examples of other suitable additives include diethyl ether (bp 35° C.), diisopropyl ether (bp 69° C.), C₄F₉OCH₃ (CAS 163702-07-6), C₄F₉OCH₂CH₃ (CAS 163702-05-4); i-C₄F₉OCH₂CH₃ (CAS 163702-06-5), and C₃F₇OCH₃ (CAS 375-03-1), as well as fluids including (CF₃)₂CFCF(OCH₃)CF₂CF₃ (73DE, CAS 132182-92-4); (CF₃)₂CFCF(OCH₂CH₃)CF₂CF₂CF₃ (HFE 7500, CAS 297730-93-9); 1,1,1,2,3,3-hexafluoro-4-(1,1,2,3,3,3-hexafluoropropoxy)pentane (HFE 7600,CAS 870778-34-0); furan,2,3,3,4,4-pentafluorotetrahydro-5-methoxy-2,5-bis[1,2,2,2-tetrafluoro-1-(trifluoromethyl)ethyl]—(HFE 7700, CAS 812-05-4), and 1,1,1,2,4,4,5,5,5-nonafluoro-(2-trifluoromethyl)-3-pentanone (Novec™ 1230, CAS 756-13-8).

The following Examples illustrate certain embodiments of the invention and shall not limit the scope of the appended claims.

### EXAMPLES

Exemplary examples of the formation of compounds of formula (2) are shown below.

### Example 1

Two-Step Reaction to Form HFO-153-10Mzz Starting from CFO-1316Mxx

HFO-153-10mzz was formed by a reaction of CFO-1316mxx with tetrafluoroethylene (TFE) to form 2,3-dichloro-1,1,1,4,4,5,5,6,6,6-decafluoro-2-hexene followed by a hydrodechlorination of 2,3-dichloro-1,1,1,4,4,5,5,6,6,6-decafluoro-2-hexene to form HFO-153-10mzz.

Reaction of CFO-1316Mxx with TFE Catalyzed by Aluminum Chlorofluoride (ACF)

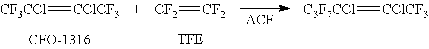

In a first reaction step, CFO-1316mxx is combined with TFE in a vapor phase reaction catalyzed by aluminum chlorofluoride to form 2,3-dichloro-1,1,1,4,4,5,5,6,6,6-decafluoro-2-hexene with a yield of at least 48%. In some embodiments, the reaction occurs under conditions described by Krespan et al., “Fluoroolefin condensation catalyzed by aluminum chlorofluoride”, Journal of Fluorine Chemistry, Vol. 77, pp. 117-126, 1996, which is incorporated by reference herein.

### Hydrodechlorination of 2,3-dichloro-1,1,1,4,4,5,5,6,6,6-decafluoro-2-hexene

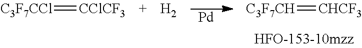

In a second reaction step, 2,3-dichloro-1,1,1,4,4,5,5,6,6,6-decafluoro-2-hexene is hydrodechlorinated in a vapor phase reaction over a palladium catalyst to form HFO-153-10mzz.

### Example 2

Two-Step Reaction to Form HFO-153-10Mzz Starting from HCFO-1326Mxz

HFO-153-10mzz can be formed by a vapor phase reaction of HCFO-1326mxz with TFE to form 2-chloro-1,1,1,4,4,5,5,6,6,6-decafluoro-2-hexene followed by a vapor phase hydrodechlorination of 2-chloro-1,1,1,4,4,5,5,6,6,6-decafluoro-2-hexene to form HFO-153-10mzz.

Reaction of HFO-1326Mxz with TFE Catalyzed by SbF₅

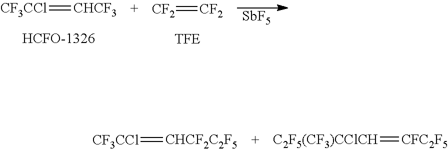

In a first reaction step, HFO-1326mxz was reacted with TFE in the presence of antimony pentafluoride (SbF₅) catalyst to form a mixture of CF₃CCl═CHCF₂C₂F₅ and C₂F₅(CF₃)CClCH═CFC₂F₅. A 400-mL Hastelloy® shaker tube was loaded with 6 g (0.028 mol) of SbF₅, shaker tube was cooled down in dry ice, evacuated and charged with 60 g (0.3 mol) of HCFO-1326mxz (CF₃CH═CClCF₃) and 50 g (0.5 mol) of TFE. It was placed in a barricade and was warmed up to ambient temperature and kept agitated for 16 hours. 100 mL of water was injected into shaker tube, it was cooled down with ice, vented off and unloaded. The organic layer was separated, dried over MgSO₄ and filtered to give 85 g of crude product, containing 50% of HCFO-1326mxz, 37% of CF₃CCl═CHC₃F₇ and 13% of 1:2 adduct of HCFO-1326/TFE (GC/MS).

The crude reaction mixture was fractionated to give 39 g (79% yield of 2-chloro-1,1,1,4,4,5,5,6,6,6-decafluoro-2-hexene, at 50% conversion of HCFO-1326mxz) of fraction boiling point 80-86° C., which was identified as a mixture of E-CF₃CCl═CHCF₂CF₃ and Z—CF₃CCl═CHCF₂CF₃ in ratio 65:35, along with 10 g of higher boiling point fraction (1:2 adduct, GC/MS).

E-CF₃CCl═CHC₃F₇:

¹⁹F NMR (CDCl₃): −70.98 (3F, s), −80.67 (3F, t, 8.9 Hz), −111.64 (2F, sixt. 10.5 Hz), −127.16 (2F, q, 4.0 Hz) ppm

¹H NMR (CDCl₃): 6.59 (t, 12.3 Hz) ppm

Z—CF₃CCl═CHC₃F₇:

¹⁹F NMR (CDCl₃): −63.64 (3F, tt, 17.7, 4.0 Hz), −80.52 (3F, t, 10.5), −107.20 (2F, m), −127.76 (2F, s) ppm¹H NMR (CDCl₃):

¹H NMR (CDCl₃): 6.41 (t, 14.5 Hz) ppm

GC/MS (m/z, mixture of isomers): 298 (M⁺, C₆HClF₁₀⁺)

1:2 Adduct—C₂F₅CF═CHCCl(CF₃)CF₂CF₃ (major isomer): bp 105-109° C. (est).

¹⁹F NMR (CDC₃): −71.83 (3F, dm, 12.1 Hz), −78.24 (3F, m), −83.96, (3F, dt, 6.1, 1.8 Hz), −108.55 (1 F, m), −115.92 (2F, AB quart., J_{d}=296.7 Hz), −122.32 (2F, ddq, 13.0, 10.8, 2.1 Hz) ppm

¹H NMR (CDC₃): 5.84 (d, 27.6 Hz) ppm

GC/MS (m/z, mixture of isomers): 398 (M⁺, C₈HClF₁₄⁺).

### Hydrodechlorination of 2-chloro-1,1,1,4,4,5,5,6,6,6-decafluoro-2-hexene

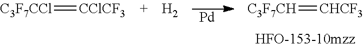

In a second reaction step, 2,3-dichloro-1,1,1,4,4,5,5,6,6,6-decafluoro-2-hexene is hydrodechlorinated in a vapor phase reaction over a palladium catalyst to form HFO-153-10mzz.

### Example 3

Three-Step Reaction to Form HFO-153-10Mczz Starting from HFC-245cb

HFO-153-10mczz can be formed by a chlorination of HFC-245cb to form CFC-215cb followed by a coupling reaction of CFC-215cb to form 3,4-dichloro-1,1,1,2,2,5,5,6,6,6-decafluoro-3-hexene followed by a hydrodechlorination of 3,4-dichloro-1,1,1,2,2,5,5,6,6,6-decafluoro-3-hexene to form HFO-153-10mczz.

### Chlorination of HFC-245cb

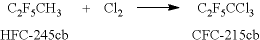

In a first reaction step, HFC-245cb is chlorinated in a vapor phase reaction to form CFC-215cb.

### Coupling of CFC-215cb

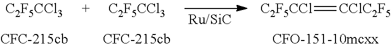

In a second reaction step, CFC-215cb couples at 130° C. in a vapor phase reaction in the presence of a 2% Ru/SiC catalyst to form 3,4-dichloro-1,1,1,2,2,5,5,6,6,6-decafluoro-3-hexene in a yield of at least 86%.

### Hydrodechlorination of CFO-151-10Mcxx

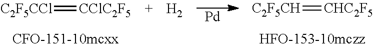

In a third reaction step, 3,4-dichloro-1,1,1,2,2,5,5,6,6,6-decafluoro-3-hexene (CFO-151-10mcxx) is hydrodechlorinated in a vapor phase reaction over a palladium catalyst to form HFO-153-10mczz.

While the invention has been described with reference to one or more embodiments, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment disclosed as the best mode contemplated for carrying out this invention, but that the invention will include all embodiments falling within the scope of the appended claims. In addition, all numerical values identified in the detailed description shall be interpreted as though the precise and approximate values are both expressly identified.

### Example 4

Hydrodechlorination of CFC-51-10Mcaa with 1% Ir/C Catalyst

10 mL of a 1% Ir/C catalyst was loaded into a 12-inch long, half-inch OD Monel 400 reactor. After an N₂ purge to remove air from the reactor, the catalyst was activated by H₂ at 250° C. for 8 hours. CFC-51-10mcaa and H₂ were then feed into the reactor at a pressure of 150 psi, an organic flowrate of 0.86 cc/hr, an H₂ flowrate of 17.9 sccm

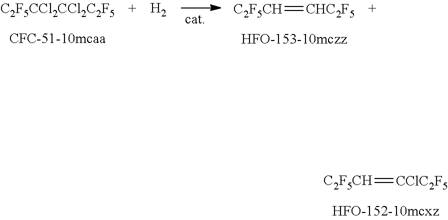

The reactor effluent was analyzed by online GC-MS-FID.

### Example 5

Synthesis of HFO-153-10 Mczz with 0.6% Pd/5.5% Cu Activated Carbon

CF₃—CF₂—CCl₂—CCl₂—CF₂—CF₃+H₂→CF₃—CF₂—CH═CH—CF₂—CF₃+CF₃—CF₂—CCl═CH—CF₂—CF₃

An Inconel (0.5 inch OD) tube reactor was loaded with 6 cc of 0.6% Pd/5.5% Cu on Activated carbon), and was pretreated with H₂ according to Table 2 below.

TABLE 2

| STEP | TIME, min. | N₂ sccm | H₂, sccm | Tube reactor, ° C. |
|---|---|---|---|---|
| 1 | 240 | 15 | 0 | 110 |
| 2 | 75 | 15 | 10.3 | 125 |
| 3 | 75 | 10 | 10.3 | 250 |
| 4 | 75 | 0 | 10.3 | 250 |
| 5 | 600 | 0 | 10 | 200 |

Liquid CFC-51-10mcaa (CF₃—CF₂—CCl₂—CCl₂—CF₂—CF₃) was fed into a heated chamber at temperature where it is vaporized and mixed with H₂ to form a CFC-51-10mcaa/H₂ reaction mixture. The reaction mixture 0.3 ml/hr CFC-51-10mcaa and 5.5 sccm H₂ were then passed through the tube reactor containing the H₂ treated Pd—Cu/C catalyst. Part of the reactor effluent was passed through a series of valves and analyzed by GCMS. Reaction conditions and results are listed in Table 3 below.

TABLE 3

| | | | | | CFC-15- | |
|---|---|---|---|---|---|---|
| HFO- | HCFO- | CFO- | | H2- | 10mcaa | Temper- |
| 153- | 152- | 151- | combined | flow | flow | ature, |
| 10mczz | 10mcxz | 10mcxx | others* | (sccm) | (ml/hr) | ° C. |
| 0.04% | 2.92% | 92.86% | 4.18% | 5.5 | 0.3 | 70 |
| 0.04% | 11.27% | 79.62% | 9.08% | 5.5 | 0.3 | 125 |
| 1.41% | 64.71% | 9.91% | 23.97% | 5.5 | 0.3 | 175 |
| 6.98% | 67.94% | 1.08% | 24.00% | 5.5 | 0.3 | 200 |
| 18.75% | 58.04% | 0.00% | 23.21% | 5.5 | 0.3 | 225 |
| 34.60% | 41.87% | 0.00% | 23.53% | 5.5 | 0.3 | 250 |
| 49.44% | 24.73% | 0.00% | 25.83% | 5.5 | 0.3 | 275 |

*combined intermediates and byproducts

Legend—CFC-51-10mcaa, CF₃—CF₂—CCl₂—CCl₂—CF₂—CF₃
HCFO-152-10mcxz, CF₃—CF₂—CCl═CH—CF₂—CF₃
CFO-151-10mcxx, CF₃—CF₂—CCl═CCl—CF₂—CF₃
HFO-153-10 mczz, CF₃—CF₂—CH═CH—CF₂—CF₃ (1,1,1,2,2,5,5,6,6,6-decafluorohex-3-ene)

### OTHER EMBODIMENTS

A working fluid comprising E-C₂F₅CF═CFC₂F₅(FO-151-12mcyyE).

A working fluid comprising E-C₂F₅CH═CHC₂F₅(HFO-153-10mczzE).

A working fluid comprising C₃F₇CH═CHCF₃ (HFO-153-10mzz).

A working fluid comprising (CF₃)₂CFCH═CHCF₃ (HFO-153-10mzzy).

A working fluid comprising Z—C₂F₅CF═CFC₂F₅ (FO-151-12mcyyZ).

Although certain aspects, embodiments and principals have been described above, it is understood that this description is made only way of example and not as limitation of the scope of the invention or appended claims. The foregoing various aspects, embodiments and principals can be used alone and in combinations with each other.

1. A method of producing a fluoroolefin comprising:
hydrodechlorinating a compound of formula (1),
CₙF₂ₙ₊₁CX₁═CClC₄₋ₙF₉₋₂ₙ (1)
wherein n is 1; and
wherein X₁ is H or Cl;
in the presence of a first catalyst and hydrogen in an amount sufficient to form a composition comprising a compound of formula (2),
CₙF₂ₙ₊₁CH═CHC₄₋ₙF₉₋₂ₙ (2);
wherein, when X₁ is Cl, the compound of formula (1) is Produced by a method comprising
contacting CF₃CCl═CClCF₃ with CF₂═CF₂ in the presence of a second catalyst in an amount sufficient to form a composition comprising the compound of formula (1); and
wherein, when X₁ is H, the compound of formula (1) is produced by a method comprising contacting CF₃CCl═CHCF₃ with CF₂=CF₂ in the presence of a second catalyst in an amount sufficient to form a composition comprising the compound of formula (1).

2. The method of claim 1, wherein the hydrodechlorinating occurs in a vapor phase.

3. The method of claim 1, wherein the first catalyst comprises a palladium catalyst.

4-6. (canceled)

7. The method of claim 1, wherein the contacting step in the method of producing the compound of formula (1) occurs in a vapor phase.

8. The method of claim 1, wherein the second catalyst comprises aluminum chlorofluoride.

9. The method of claim 8, wherein the aluminum chlorofluoride has the formula AlClₓF₃₋ₓ, wherein x is in the range of 0.05 to 0.3.

10-18. (canceled)

19. The method of claim 39, wherein the coupling occurs in a vapor phase.

20. The method of claim 39, wherein the coupling is performed at a temperature of about 130° C.

21. The method of claim 39, further comprising:
chlorinating C₂F₅CH₃ in the presence of a second catalyst comprising SbF₅, in an amount sufficient to form a composition comprising C₂F₅CCl₃.

22-24. (canceled)

25. A composition formed by the method of claim 1.

26-27. (canceled)

27. (canceled)

28. A composition formed by the method of claim 39.

29. (canceled)

30. A method of producing a fluoroolefin comprising: hydrodechlorinating a compound of formula (3), CₙF₂ₙ₊₁CCl₂CCl₂ C₄₋ₙF₉₋₂ₙ(3) wherein n is 1 or 2; in the presence of a first catalyst and hydrogen in an amount sufficient to form a composition comprising a compound of formula (2), CₙF₂ₙ₊₁CH═CHC₄₋ₙF₉₋₂ₙ(2).

31. A composition comprising CF₂═CF₂, CF₃CCl═CClCF₃ and Z—CF₃—CH═CH—CF₃.

32. A composition comprising CF₂═CF₂, CF₃CCl═CClCF₃, a catalyst and optionally Z—CF₃—CH═CH—CF₃.

33. The composition of claim 32, wherein the catalyst comprises aluminum chloride (AlCl₃), antimony pentafluoride (SbF₅), or aluminum chlorofluoride defined by the formula AlClₓF₃₋ₓ or antimony chlorofluoride of formula SbClₓF₅₋ₓ, where x is one of an integer from 1 to 3 or a decimal of from 0.01 to 0.5.

34. (canceled)

35. A composition comprising CF₃—CF₂—CH═CH—CF₂—CF₃ (HFO-153-10mczz), and optionally one or more of CF₃—CF₂—CCl═CCl—CF₂—CF₃ (CFO-151-10mcxx), CF₃—CF₂—CCl═CH—CF₂—CF₃ (HCFO-152-10mcxz) and CF₃—CF₂—CCl₂—CCl₂—CF₂—CF₃ (CFC 51-10mcaa).

36. A composition comprising between 1 and 50 mole % CF₃—CF₂—CH═CH—CF₂—CF₃ (1,1,1,2,2,5,5,6,6,6-decafluorohex-3-ene) and between about 3 to 70 mole percent CF₃—CF₂—CCl═CH—CF₂—CF₃ (HCFO-152-10mcxz) wherein the total amount of HFO-153-10mzcc and HCFO-152-10mcxz is between 3 mole percent and 75 mole percent.

37. A product stream comprising based on to total product stream content between greater than 0 and 50 mole % CF₃—CF₂—CH═CH—CF₂—CF₃ (1,1,1,2,2,5,5,6,6,6-decafluorohex-3-ene) and between about 3 to 70 mole percent CF₃—CF₂—CCl═CH—CF₂—CF₃ (HCFO-152-10mcxz), wherein the total amount of HFO-153-10mczz and HCFO-152-10mcxz is between 3 mole percent and 75 mole percent.

38. A mixture comprising a compound of formula CₙF₂ₙ₊₁CX₁═CClC₄₋ₙF₉₋₂ₙ (1) wherein X₁ is H or Cl, a compound of formula CₙF₂ₙ₊₁CH═CHC₄₋ₙF₉₋₂ₙ (2) wherein in formula (1) and (2) n=1 or 2, and optionally a catalyst comprising one of a Lewis acid or metal comprising at least supported or unsupported palladium.

39. A method of producing a fluoroolefin comprising:
hydrodechlorinatina a compound of formula (1),
CₙF₂ₙ₊₁CX₁═CClC₄₋ₙF₉₋₂ₙ (1)
wherein n is 2; and
wherein X₁ is Cl;
in the presence of a first catalyst and hydrogen in an amount sufficient to form a composition comprisinq a compound of formula (2),
CₙF₂ₙ₊₁CH═CHC₄₋ₙF₉₋ₙ (2);
wherein, the compound of formula (1) is produced by a method comprising
coupling C₂F₅CCl₃ under conditions sufficient to form a composition comprising the compound of formula (1).
